(19) United States
(12) Patent Application Publication
(10) Pub. No.: US 2008/0317733 A1
(43) Pub. Date: Dec. 25, 2008

(54) Cosmeceutical Compositions and Methods with Biologically Active Ingredients

(75) Inventor: Nazli Azimi, San Juan Capistrano, CA (US)

Correspondence Address: AZIMI NAZLI, 27462 PASEO BOVEDA, SAN JUAN CAPISTRANO, CA, 92675, US

(73) Assignee: NuGENE INC.

(21) Appl. No.: 11/766,027

(22) Filed: Jun. 20, 2007

(51) Int. Cl. A61K 38/00; A61K 31/195; A61K 38/48; C12Q 1/68; G01N 33/53; A61P 17/00; A61K 31/70

(52) U.S. Cl. 424/94.63; 435/6; 435/7.1; 514/12; 514/18; 514/23; 514/561

(57) ABSTRACT

Cosmeceutical formulations are personalized to address an individual's nutrient deficiencies and genetic predispositions for premature aging of the skin. Growth factors and high doses of inefficiently absorbed nutrients are nanoencapsulated and delivered topically to improve the function, health, and appearance of skin and hair.

### RELATED APPLICATIONS

This application claims the benefit of and priority to U.S. Utility patent application Ser. No. 11/765,977 filed 20 Jun. 2007, entitled “Personalized Nutrient Deficiency Assay” the contents of which are incorporated by reference herein in its entirety.

### BACKGROUND

The present disclosure relates to skin care compositions for providing treatment, restoration, maintenance, and protection of skin.

### SUMMARY

Cosmeceutical formulations are personalized to address an individual's nutrient deficiencies and genetic predispositions for premature aging of the skin. Growth factors and high doses of inefficiently absorbed nutrients are nanoeneapsulated and delivered topically to improve the function, health, and appearance of skin and hair.

According to a feature of the present disclosure, a method is disclosed comprising providing a personalized nutrient and genetic screening to identify nutrient deficiencies and genetic variations, and providing a cosmeceutical with at least one active ingredient to address the deficiencies. The active ingredient comprises at least one vitamin, mineral, growth factor, peptide fragment of growth factors, antioxidant, protease, natural extract, or amino acid.

According to a feature of the present disclosure, a composition is disclosed comprising a cosmeceutical formulation having at least one active ingredient. The cosmeceutical formulation is formulated to address at least one nutrient deficiency, the nutrient deficiency determined by at least one personalized nutrient screen process. Moreover, the active ingredient further comprises at least one vitamin, mineral, growth factor, peptide fragment of growth factors, antioxidant, protease, natural extract, or amino acid.

According to feature of the present disclosure, a method comprising providing a personalized nutrient screening to identify nutrient deficiencies, correlating the observed deficiencies to a database of growth factors to determine a set of growth factors that may be administered to overcome the deficiencies, and providing a cosmeceutical with at least one active ingredient to address the deficiencies. The active ingredient comprises at least one vitamin, mineral, growth factor, peptide fragment of growth factors, antioxidant, or amino acid and the cosmeceutical is prepared from a base cosmeceutical formulation and a plurality of deficiency-based cosmeceutical formulations tailored to address various nutrient deficiencies.

### DRAWINGS

The above-mentioned features and objects of the present disclosure will become more apparent with reference to the following description taken in conjunction with the accompanying drawings wherein like reference numerals denote like elements and in which:

FIG. 1 is a cross-sectional view of an embodiment of a nanosome having at least one biologically active ingredient:

FIG. 2 is a side view of an embodiment of a method of improving the health of skin using a cosmeceutical; and

FIG. 3 is a block diagram of an embodiment of a method of creating a personalized cosmeceutical regimen.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may he made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

As used in the present disclosure, the term “active ingredient” shall he defined as a component of a cosmeceutical that interacts with or effects changes in tissues, cells, biological pathways, or native molecules.

As used in the present disclosure, the term “cosmeceutical” shall be defined as a substance that is applied topically to an organism. Cosmeceuticals include cosmetics with pharmaceutical properties and pharmaceutics that are applied topically.

The present disclosure relates to cosmeceuticals having nanosome encapsulated growth factors, vitamins, minerals, amino acids, anti-oxidants, and other active ingredients to increase the health and function of skin and hair, as well as reverse the effects of aging with respect to skin appearance.

The body comprises a complex system of biological pathways. A single base pair mutation, also known as small nucleotide polymorphisms, may cause a breakdown or an inefficiency in a biological pathway that results in a deficiency in the production of growth factors or uptake of nutrients.

Of the body's organs, the skin shows the effects of “wear and tear” on the organism as it is exposed to UV radiation and other harsh environmental conditions. The appearance of a person is greatly impacted by the combination of “wear and tear” and breakdowns or inefficiencies in the production of growth factors and uptake of nutrients.

According to the present disclosure, cosmeceuticals containing active ingredient are disclosed. The cosmeceutical contains at least one or more biologically active ingredients based on the desired function of the cosmeceutical. The ingredients may be encapsulated in nanosomes depending on the susceptibility of each active ingredient to degradation within the cosmeceutical. Multiple ingredients may be encapsulated together within single nanosomes or the ingredients may be individually encapsulated. Generally, the active ingredients are mixed in a straightforward manner with the base cosmeceutical.

The active ingredients include but are not limited to proteins, peptides, proteases, vitamins, minerals, fatty acids, amino acids, nucleic acids, and anti-oxidants. In particular, the proteins include growth factors such as EGF, IGF-1, bFGF, aFGF, KGF, TGF-β₃, Thymosin β₄. and VEGF. Additionally, thioredoxin, peptide TGP2, copper peptide, ascorbic acid, kojic acid, glycolic acid, hyaluronic acid, arbutin, coenzyme Q10, licorice extract, oryza, sative hull extract, green tea extract, titanium, dioxide, and zinc oxide may also be used.

The active ingredients may include chemically synthesized peptides which mimic the function of naturally produced growth factors in a human body.

Other ingredients may be added to the cosmeceuticals that do not have biologically active properties. These ingredients include, for example, moisturizing ingredients, ingredients that impart desired properties such a UV resistance, and ingredients that impart the characteristics of the cosmeceutical, such as color, consistency, texture, viscosity, and smell.

According to various embodiments, the cosmeceuticals may be in various embodiments and forms including creams, lotions, moisturizers, toners, gels, cleansers, masks, sprays, gels, and solutions. According to embodiments, the cosmeceutical comprises a patch that releases active ingredients encapsulated in nanosomes through the skin. According to embodiments, the cosmeceuticals include foaming cleansers, moisturizers, toners, day creams, night creams, eye creams, lotions, sun screens and sun blocks, serums, and healing creams.

Desired functions of the cosmeceuticals include, modulating skin cell metabolism and function, repairing aged or damaged skin, skin nourishing and moisturizing, improving skin elasticity and plasticity, skin smoothing, and protection of skin against radicals and UV rays, according to embodiments.

According to embodiments, the uptake of nutrients is improved by packaging the nutrients into nanosomes, which are readily absorbed through the skin and the contents thereof delivered intracellularly. The skin is designed, to be impervious to water. Because many nutrients are water soluble, nutrients are inefficiently absorbed through the skin. By encapsulating water soluble nutrients, growth factors, etc., into nanosomes, they may be absorbed through the skin and into the target cells. Similarly, fat soluble nutrients and growth factors are storable and deliverable using nanosomes as well.

According to embodiments illustrated in FIG. 1, nanosomes 10, which, are essentially small (˜30 nm) liposomes, are formed from a bilayer of molecules having hydrophobic head 18 and hydrophilic tail 16. For example, a nanosome may be constructed having a phospholipid and cholesterol bilayer. Like cells, the phospholipids aggregate together to form a “capsule” with the hydrophobic portion of the bilayer on the outside and the hydrophilic portion on the inside, much as oil micelles form in water. Encapsulated in nanosome 10 are one or more active ingredients 20 to be delivered.

Nanosomes may be desirable, according to embodiments, because they improve bioavailability of the active ingredients encapsulated within them. Bioavailability is increased because more active ingredient is actually delivered to its target. Moreover, nanosomes readily cross through the gastrointestinal membrane, thereby effectively transporting the active ingredients into the blood stream. Additionally, because the bioavailability is increased, less active ingredient must be used, which reduces the costs associated with producing and delivering active ingredients to effect a result. Because the active ingredients encapsulated in the nanosome are protected from the environment, the active ingredients have a longer shelf life and are more stable on average than those not encapsulated. Importantly, active ingredients encapsulated within a nanosome can undergo pasteurization without destroying the active ingredients in the process.

Most importantly, however, the nanosome protects the active ingredients from oxidation, hydrolysis, reduction, and enzymatic reaction, thereby allowing the active ingredient to arrive at its target in an active state. Finally, nanosome encapsulation masks odor and taste of the active ingredients, making them more pleasing to those who use the active ingredients.

The growth factors of the present disclosure may be encapsulated in nanosomes, and the nanosomes put into a delivery vehicle. Thus, they will be effectively delivered through the skin or into the blood stream. Thereafter, the nanosomes may be absorbed into cells by fusing with cellular membranes or via endocytosis, for example.

Nanosomes are excellent tools for delivering pharmacological active ingredients through the skin or the digestive tract directly to the cell without exposing the active ingredients to degradation events prior to delivery. Additionally, nanosomes allow active ingredients that would otherwise only inefficiently arrive at the target cells, for example, water soluble active ingredients applied topically. Thus, in combination, with the novel active ingredients disclosed herein, nanosomes provide a delivery device to efficiently cause the active ingredients to be delivered intracellulary.

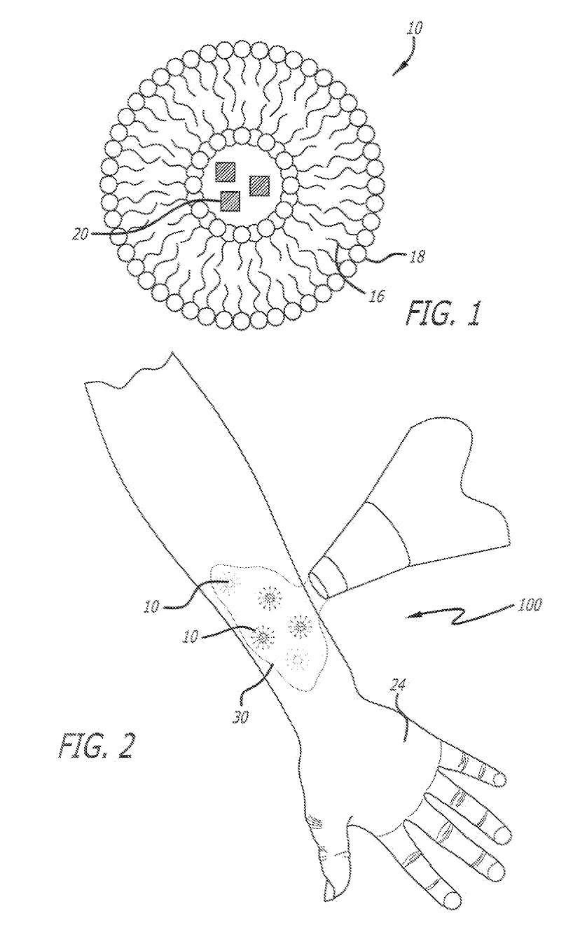

According to embodiments and as illustrated in FIG. 2, the active ingredients of cosmeceuticals are encapsulated in nanosome 10 and disposed, in a variety of delivery vehicles 30. For example, any of copper peptides; growth factors including: EFG, KGF, TGF-β₃, thymosin β₄, IGF-1, aFGF, bFGF, VEGF; peptide sequences that mimic or enhance the activity of proteins; proteases; thioredoxin; titanium dioxide; zinc oxide; arbutin, coenxyme Q10; licorice extract; oryza; sative hull extract; green tea extract; kojic acid; glycolic acid; hyaluronic acid; vitamins; minerals; and amino acids are encapsulated into nanosomes, and nanosomes 10 put into delivery vehicles 30, such as hair shampoos, hair conditioners, hair sprays, massage oils, and topic serums for direct delivery to the areas affected, by or predisposed to hair loss. Indeed, because nanosomes 10 are stable in a water based solutions, nearly any such solution that can be applied to hair loss areas forms an effective delivery mechanism for nanosomes having the hair loss prevention or hair growth active ingredients. Delivery vehicle 30 is applied in process 100 the skin 24 to effect delivery of the active ingredients. Likewise, delivery vehicle 30 may be pills, beverages, and other ingestibles for delivery of nanosomes 10 encapsulating hair loss prevention and hair growth active ingredients into the blood stream.

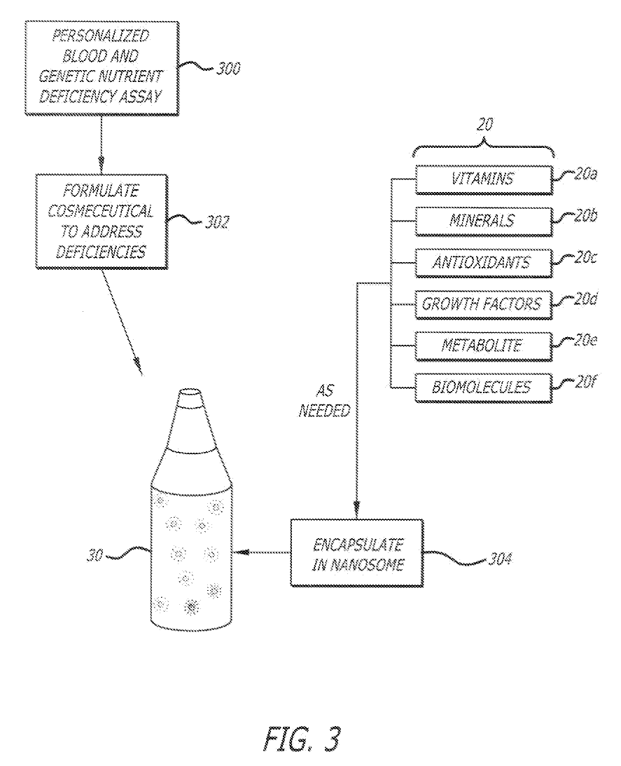

According to embodiments and as illustrated in FIG. 3, personalized cosmeceutical regimens are provided that comprise a balance of growth factors and nutrients deficient in a particular individual. Accordingly, an individual will undergo testing, such a blood assays, genetic assays, hormone assays, skin assays, etc. to determine the individual's deficiencies in process 300. After the deficiencies are determined, individual cosmeceutical formulations are formulated in process 302 and administered. According to embodiments, the active ingredients 20 are encapsulated in nanosomes in process 304, for example, to deliver intracellularly the growth factors and nutrients necessary for full health of the skin. For example, a patient might be deficient in one or more of vitamins 20A, minerals 20B, antioxidants 20C, growth factors 20D, metabolites 20E and biomolecules 20F, which may be individually dosed to overcome the deficiency or correct the biological inefficiency in uptake. The personal formulation 30 may then be applied by the patient to address the deficiencies, for example as shown in FIG. 2. Application of cosmeceuticals is well known by artisans and users. According to embodiments, active ingredients may also be suspended in a base cosmeceutical formulation without encapsulation.

For example, where a gene mutation interrupts a biological process or reduces the efficiency of a process such that a given growth factor is inefficiently produced, providing the growth factor from an external source bypasses the genetic anomaly allowing the cells to uptake the nutrient at the same level as is within a normal range in a population. Similarly, where the a nutrient uptake is inefficient, for example, due to a breakdown in the uptake biological processes, an abundance of that nutrient may be provided as a “brute force” mechanism to overcome the inefficiency. Delivery by nanosomes provides a platform for delivery into cells without degradation, inactivation, oxidation, or reduction of the active ingredients.

According to embodiments tissue, blood, and serum tests measuring quantities of an individual's nutrients are used to provide information on deficiencies that may be associated with an individual's diet and uptake of nutrients. Diet and nutrient intake is one of the many factors that influence the nutritional status of an individual. Insufficient intake or uptake of specific nutrients generally results in a deficiency of that nutrient.

According to embodiments, the nutritional deficiency assay is an assay measuring levels of accumulation of the nutrient in lymphocytes of the individual. Generally, an intracellular function assay is used and comprises the steps of collecting lymphocyte cells, isolating the cells from other whole blood components, and maintaining the cells in culture during the assay. The lymphocytes collected have a 4-to 6-month lifespan and are generally in a resting state in terms of cell division. The resting lymphocytes are stimulated to undergo cell division and grow in culture. The degree of growth that the lymphocytes are able to maintain, following stimulation is directly related to the nutrient levels accumulated in the lymphocytes. From the lymphocyte degree of growth, a functional intracellular analysis of nutrient status may be obtained.

According to embodiments, the nutrient targeted in the nutritional deficiency assay is selected from vitamins, minerals, amino acids, antioxidants, and metabolites. According to embodiments, as shown in Table 1, the nutrient is a vitamin such as vitamin A, B1, B2, B₃, B6, B12, D, E, biotin, folate, and panothenate; minerals such as calcium, magnesium, selenium, and zinc; an amino acid such as asparagine, carnitine, glutamine, and serine; an antioxidant selected from coenzyme Q10, glutathione, and cysteine; or a metabolite such as lipoic acid, oleic add, choline, inositol, fructose, glucose, and insulin. Other vitamins, minerals, antioxidants, and nutrients may also be targeted in the nutritional deficiency assay, as would be known, and understood by artisans.

TABLE 1

| Nutrient | |
|---|---|
| Type | Specific Nutrient Tested |
| Vitamin | vitamin A, B1, B2, B3, B6, B12, D, E, biotin, folate, |
| | panothenate |
| Mineral | calcium, magnesium, selenium, zinc |
| Amino Acid | asparagine, carnitine, glutamine, serine |
| Antioxidant | coenzyme Q10, glutathione, cysteine |
| Metabolite | lipoic acid, oleic acid, choline, inositol, fructose, |
| | glucose, insulin |

According to embodiments, genetic testing is used to provide information regarding an individual's health based on the individual's genetic conditions. An individual's inherited health risks and potential health problems may be assessed through the genetic testing. More importantly, correlations maybe drawn to nutritional deficiencies based on observed genetic mutations. Thus, genetic assays may lead, to information regarding the cause of nutrient deficiencies or nutritional deficiencies that are unobserved in other assay method. This allows for the development of a suitable diet, lifestyle, and supplement regimen that matches the unique health risks of an individual's body.

In one embodiment, the genetic variation assay is an assay searching for single nucleotide polymorphisms (SNPs) in the gene. A SNP is a natural variation or change in a single nucleotide base pair of a gene, which may affect the way an individual's body responds to certain stimuli such as damage, infection, or even food intake. Cells are collected and select segments of the genetic code are tested for SNPs. The cells are preferably collected from a mouth rinse procedure, but can also be collected from a blood sample or other methods. The genetic test evaluates SNPs that may lead to health problems when influenced by other external and environmental factors. Based on the genetic test results, a customized treatment plan may be developed.

Functional assessment tests may also be conducted to monitor if an individual's personalized health regimen has an impact on the individual's genetic risks. By evaluating how the individual's body responds to treatment, functional assessment tests may be used to ensure that environmental factors, such as hormones and nutrients, are in a state of optimal balance that minimizes the individual's in-born genetic health risks.

According to embodiments, the gene assayed is at least one of TNFα, IL-6, IL-1β, SOD1, SOD2, CAT, EPHX1, GPX1, MnSOD2, p53, IGF, TGFβ1, MMP, EGF, ELA2, VEGF, bFGF, APOE, MTHFR, and ACE. Genes evaluated include but are not limited to the genes shown in the following Table 2.

TABLE 2

| Gene | Full Name | SNP Location |
|---|---|---|
| TNFα | tumor necrosis factor α | −308G/A |
| IL-6 | interleukin 6 | −174C/G |
| IL-1β | interleukin 1β | −31C/T |
| SOD1 and SOD2 | superoxide dismuatse 1 and 2 | 47C/T |
| CAT | Catalase | −262G/A |
| EPHX1 | microsomal epoxide hydrolase 1 | 113T/C |
| GPX1 | Glutathione peroxidase | 593C/T |
| MnSOD2 | Manganese superoxide dismuatse | 399T/C |
| P53 | Tumor suppressor | 72 codon Proline |
| | | to Arginine |
| IGF | Insulin Like growth factor | 192 bp allele of |
| | | IGF1 promoter |
| TGFβ1 | transforming growth factor β1 | +29T/C |
| MMP | Matrix metelloproteinase | −160G/GG |
| EGF | Epidermal growth Factor | 61G/A |
| ELA2 | Neutrophil elastase | −903T/G and |
| | | −714G/A |
| VEGF | Vascular Endothelial growth factor | 405G/C |
| bFGF | Basic Fibroblast Growth Factor | −553T/A |
| APOE | apolipoprotein E | 388C/T |
| MTHFR | methylenetetrahydrofolate | 776C/G |
| | reductase | |
| ACE | angiotensin I-converting enzyme | Insertion/deletion |
| | | at intron 16 |

According to embodiments, the method for providing a health regimen comprises formulating at least one cosmeceutical based upon at least one of the nutritional deficiency assay result and genetic variation assay result Active ingredients include, vitamins, minerals, growth factors, antioxidants, metabolites, amino acids, nucleic acids, and other biomolecules, etc. to address specific nutrient deficiencies of the individual if a nutrient is inefficiently absorbed, higher doses of the nutrient may correct the deficiency in a “brute force” manner, or the machinery for uptake of the nutrient may he repaired to increase uptake, according to embodiments.

According to embodiments, a health regimen is provided for an individual. The health regimen comprises: (1) at least one nutritional deficiency assay targeting at least one nutrient of the individual, wherein the nutritional deficiency assay produces a nutritional deficiency assay result; (2) at least one genetic variation assay targeting at least one gene of the individual, wherein the genetic variation assay produces a genetic variation assay result; and (3) at least one health recommendation to the individual, wherein the health recommendation is based upon at least one of the nutritional deficiency assay result and genetic variation assay result.

According to embodiments, each customized personal formula may be selected from, a preproduced formulation of active ingredients. These “custom” formulas are recommended to each Individual based on their unique nutritional needs. The individual formula may be selected from a library of nutraceutical formulas based on observed deficiencies and needs across a population.

The library is created by collecting a database of patients who have gone through a given testing process. Their test results determine the nutritional deficiencies of each individual. After statistical analysis of their deficiencies across the population of patients tested, a pattern is determined. The deficiency patterns may be correlated to gene mutations, hormonal and neurotransmitter imbalances, and phenotypic traits, for example to further identify and fine tune the assay process.

According to embodiments, a statistical approach for determining deficiencies applies a normal curve to the population. Those having nutritional deficiencies will be observed outside the confidence levels of the normal curve. As the statistical analysis is applied for larger populations, patterns will emerge wherein certain deficiencies are linked, for example a deficiency in the uptake of calcium with a deficiency in vitamin D. Thus, formulations may be designed to address each deficiency from the standpoint of providing all of the necessary active ingredients to address a given deficiency.

To the extent that patterns may be determined, formulations are preproduced with a formulation of active ingredients to address identified deficiencies. According to embodiments, the preproduced formulations may be added to a base cosmeceutical, for example a lotion. If multiple deficiencies are observed, multiple formulations may be added to the base cosmeceutical provided no one active ingredient is dosed at a dangerous level.

Similarly, more general formulas are formulated, according to embodiments, to less specifically address with deficiencies. Users may undergo the assay process to accurately Identify deficiencies or may consult another source to determine potential deficiencies, such as a doctor or a table detailing deficiencies based on diet. The user then obtains one or more preformulated cosmeceuticals to address the deficiencies, wherein the preformulated cosmeceutical comprises a personal regimen only insofar as the selected preformulation includes the necessary ingredients to treat the deficiencies. For example, a set of formulations may be devised as follows, with dosages as shown in Tables 1-4:

Formulation A: High dose vitamins+High dose anti-oxidants+RDA dose minerals

Formulation B: High dose vitamins+RDA dose anti-oxidants+RDA dose minerals

Formulation C: RDA dose vitamins+High dose anti-oxidants+RDA dose minerals

Formulation D: RDA dose vitamins+High dose anti-oxidants+High dose minerals

Formulation E: High dose vitamins+High dose anti-oxidants+Amino Acids

Formulation F: High dose vitamins+RDA dose anti-oxidants+Amino Acids

Formulation G: RDA dose vitamins+RDA dose anti-oxidants+High dose minerals+Amino Acids

TABLE 3
Vitamins (*based on caloric intake)

| Ingredient | High dose | RDA dose |
|---|---|---|
| Vitamin B1 (Thiamin) | 30-60 mg daily | 0.5-2 mg daily* |
| Vitamin B2 (Riboflavin) | 30-60 mg daily | 0.6-2.0 mg daily* |
| Vitamin B3 (Niacinamide) | 30-50 mg daily | 6.6-25 mg daily* |
| Vitamin B6 (Pyridoxine) | 25-60 mg daily | 1.6-2.0 mg daily* |
| Vitamin B12 (Cobalamin) | 100-400 mcg daily | 2-5 mcg daily* |
| Folic acid | 500-600 mcg daily | 180-400 mcg daily |
| (Methyltetrahydrofolic acid) | | |
| Vitamin B5 (Pantothenate) | 60-100 mg daily | 4-7 mg daily |
| Biotin | 100-400 mcg | 30-100 mcg daily |
| Beta-carotene | 10,000-15,000 IU | not established |
| Vitamin A | 2500-5000 IU | 2500-5000 IU |
| Vitamin C | 300-500 mg | 60-100 mg |
| Vitamin D | 400-600 IU | 200-400 IU |
| Vitamin E (D-α-tocopherol) | 100-400 IU | 12-30 IU |
| Vitamin K | 85-100 mcg daily | 65-85 mcg daily |

TABLE 4
Antioxidants (*RDA dosage not established)

| Ingredient | High dose | RDA dose |
|---|---|---|
| Glutathione | 750-1000 mg N-Acetyl- | 75-100 mg N-Acetyl- |
| | Cysteine daily | Cysteine daily |
| Co-Enzyme Q10 | 50-70 mg daily | 5-7 mg daily* |
| Selenium | 600-800 mcg daily | 60-80 mcg daily |
| Vitamin E (α- | 50-70 IU d-α-tocopherol | 35-45 IU d-α-tocopherol |
| tocopherol) | | |
| Vitamin C | 900-1100 mg daily | 90-110 mg daily |
| Alpha Lipoic Acid | 400-700 mg daily | 40-70 mg daily* |

TABLE 5
Minerals (*RDA dosage not established)

| Ingredient | High dose | RDA dose |
|---|---|---|
| Calcium Citrate | 400-1500 mg daily | 200-300 mg daily |
| Zinc Citrate | 140-160 mg daily | 14-16 mg daily |
| Magnesium | 200-400 mg daily | 150-200 mg daily |
| Boron | 1-2 mg | 0.5-1 mg* |
| Chromium | 130-160 mcg | 100-130 mcg |
| Copper | 3-4 mg | 1-2 mg |
| Iron | 20-100 mg | 10-18 mg |
| Manganese | 5-10 mg | 2-5 mg |
| Molybdenum | 120-150 mcg | 75-100 mcg |
| Potassium | 300-500 mg | 50-100 mg (only 2-3% of |
| | | the RDA) |
| Selenium | 200-300 mcg | 50-80 mcg |
| Vanadium | 10-150 mcg | 5-10 mcg* |
| Iodine | 150-300 mcg | 100-170 mcg |

TABLE 6
Amino Acids (*RDA dosage not established)

| Ingredient | Dose* |
|---|---|
| L-Phenylalanine | 200-500 mg |
| L-Lysine HCl | 200-500 mg |
| L-Valine | 200-500 mg |
| L-Leucine | 200-500 mg |
| L-Isoleucine | 200-500 mg |
| L-Methionine | 200-500 mg |
| L-Threonine | 200-500 mg |
| Glycine | 200-500 mg |
| L-Glutamine | 200-500 mg |
| L-Arginine (as L- | 200-500 mg |
| arginine HCl) | |
| L-Serine | 200-500 mg |
| L-Alanine | 200-500 mg |
| L-Aspartic acid | 200-500 mg |
| L-Histidine | 200-500 mg |
| L-Proline | 200-500 mg |
| N-Acetyl L-cysteine | 200-500 mg |
| L-Tyrosine | 200-500 mg |

According to other embodiments, static cosmeceutical formulations are similarly appropriate with emphasis on the end result of each formulation. For example, a cosmeceutical may be provided for elderly users designed to restore elastins and collagens in the skin, thereby reducing lines and wrinkles by improving the “tightness” of the skin. Conversely, a healing cosmeceutical may comprise growth factors and active ingredients specifically designed for providing skin around wounds with the necessary active. Ingredients for efficient wound healing with minimal scar tissue.

The following examples show formulations that have been shown to be effective for various cosmeceuticals and their related functions. It will he appreciated that the active ingredients of one are applicable to others, depending on the specific aims of the given formulation. For example, a daily moisturizer/skin cream may have titanium dioxide and zinc oxide added to impart UV resistance, etc.

### EXAMPLE 1

According to embodiments of the present disclosure, a cosmeceutical is a skin cleanser for removing make-up, dirt, and debris from the skin, as well as other impurities. The skin cleanser, according to embodiments, comprises a detergent and a moisturizing active ingredient, whereby the skin is cleansed and moisturized simultaneously. Included in the skin cleansing composition, according to an embodiment, are proteases, thioredoxin or other antioxidants, vitamins, and amino acids.

### EXAMPLE 2

According to embodiments, a cosmeceutical is a moisturizer or a skin cream. The moisturizer/skin cream is designed to prevent wrinkles, induce the generation of new skin cells, and increase skin elasticity. The active ingredients according to the exemplary embodiment comprise EGF, IGF-1, bFGF, thioredoxm, aFGF, copper peptide, peptides that mimic the function of the growth factors and other proteins, vitamins, minerals, and amino acids.

### EXAMPLE 3

According to embodiments, a cosmeceutical of the present disclosure comprises lotions. The lotions are as known in the art, according to embodiments, except have nanosomes encapsulating active ingredients, for example growth factors, antioxidants, etc. that are included in the lotion to deliver the active ingredients to the intracellular skin targets. According to embodiments, active ingredients include peptide TGP2, copper peptide, ascorbic acid, artutin, coenzyme Q10, licorice extract, oryza, sative hull extract, and green tea extract.

### EXAMPLE 4

According to embodiments, a cosmeceutical of the present disclosure comprises a sunscreen. In addition to active ingredients well known to artisans for absorbing UV radiation such as titanium dioxide and zinc oxide, the sunscreen may also comprise nanosomes encapsulating EGF and copper peptide, according to embodiments.

### EXAMPLE 5

According to embodiments, a cosmeceutical of the present disclosure comprises an exfoliator. The exfoliator is designed to remove dead skin cells and induce health in the newly exposed layer. According to embodiments, the active ingredients include thioredoxin, proteases, kokic acid, glycolic acid, vitamins, and amino acids.

### EXAMPLE 6

According to embodiments, a cosmeceutical of the present disclosure comprises a skin serum for the elderly. It contains growth factors, vitamins, and antioxidants to improve the appearance of aged skin, such as wrinkles and lines by increasing the level of collagen, elastin, and the extracellular matrix generally. According to embodiments, nanosomes encapsulate a high concentration of growth factors, that among other functions, induces growth of the extracellular martix including EGF, IGF-1, bFGP, thioredoxin, aFGF, copper peptide, peptides mimicking the function of the growth factor proteins, vitamins, minerals, and amino acids.

### EXAMPLE 7

According to embodiments, a cosmeceutical of the present disclosure comprises a healing cream or ointment that induces more rapid healing of wounds and reducing the residual scar tissue. According to embodiments, the active Ingredients include nanosome encapsulated copper peptide, EGF, bFGF, TGF-β₃, and hyaluronic acid.

While the apparatus and method have bees described in terms of what are presently considered to be practical embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
providing a personalized nutrient screening to identify nutrient deficiencies:
providing a cosmeceutical with at least one active ingredient to address the deficiencies;
wherein the active ingredient comprises at least one vitamin, mineral, growth factor, peptide fragment of growth factors, antioxidant, protease, natural extract, or amino acid.

2. The method of claim 1, wherein the growth factors comprise, bFGF, TGF-β₃, hyaluronic acid, aFGF, KGF, TGF-β₃, TRX, copper peptide, kokic acid, and glycolic acid.

3. The method of claim 2, wherein the copper peptide is at least one of the copper containing peptides selected from the group consisting of: Cu(II)-His-Val-His, His-Ala-His-Cu(II), and Gly-His-Lys-Cu(II).

4. The method of claim 1, wherein the screening comprises at least one of a blood assay to determine the levels of nutrients within lymphocytes and a genetic assay to observe genetic mutations giving rise to nutrient deficiencies.

5. The method of claim 4, wherein the deficiencies are correlated to a database of growth factors that are known to reduce the deficiencies.

6. The method of claim 4, wherein the screening further comprises a hormonal assay.

7. The method of claim 1, wherein the cosmeceutical is prepared from a base cosmeceutical formulation and a plurality of cosmeceutical formulations tailored to address various nutrient deficiencies.

8. The method of claim 7, wherein the base cosmeceutical formulation comprises one of a lotion, a skin cream, a cleanser, an exfoliator, a moisturizer, a sunscreen, a toner, a gel, a patch, and a healing cream or ointment.

9. The method of claim 7, wherein multiple cosmeceutical formulations tailored to address various nutrient deficiencies are included together with the base cosmeceutical formulation in the cosmeceutical.

10. The method of claim 1, wherein the cosmeceutical is one of a plurality of pre-formulated cosmeceuticals.

11. The method of claim 10, wherein the pre-formulated cosmeceuticals comprise a pharmaceutical selected from the group consisting of:
High dose vitamins+High dose anti-oxidants+RDA dose minerals;
High dose vitamins+RDA dose anti-oxidants+RDA dose minerals;
RDA dose vitamins+High dose anti-oxidants+RDA dose minerals;
RDA dose vitamins+High dose anti-oxidants+High dose minerals;
High dose vitamins+High dose anti-oxidants+Amino Acids;
High dose vitamins+RDA dose anti-oxidants+Amino Acids;
RDA dose vitamins+RDA dose anti-oxidants+High dose minerals+Amino Acids.

12. The method of claim 1, wherein the cosmeceutical is formulated on a per individual basis, whereby each individual's cosmeceutical formulation is determined based on the individual's nutritional deficiencies.

13. A composition comprising:
a cosmeceutical formulation having at least one active ingredient:
wherein the cosmeceutical formulation is formulated to address at least one nutrient deficiency, the nutrient deficiency determined by at least one personalized nutrient screen process; and
wherein the active ingredient further comprises at least one vitamin, mineral, growth factor, peptide fragment of growth factors, antioxidant, protease, natural extract, or amino acid.

14. The composition of claim 13, wherein the growth factors comprise. bFGF, TGF-β₃, hyaluronic acid, aFGF, KGF, TGF-β₃, TRX, copper peptide, kokic acid, and glycolic acid.

15. The method of claim 14, wherein the copper peptide is at least one of the copper containing peptides selected from the group consisting of: Cu(II)-His-Val-His, His-Ala-His-Cu(II), and Gly-His-Lys-Cu(II).

16. The composition of claim 13, wherein the personalized nutrient screening process comprises at least one of a blood assay to determine the levels of nutrients within lymphocytes and a genetic assay to observe genetic mutations giving rise to nutrient deficiencies.

17. The composition of claim 16, wherein the personalized nutrient screening process further comprises a hormonal assay.

18. The composition of claim 13, wherein the cosmeceutical formulation is prepared from a base cosmeceutical formulation and a plurality of deficiency-based cosmeceutical formulations tailored to address various nutrient deficiencies.

19. The composition of claim 18, wherein the base cosmeceutical formulation comprises one of a lotion, a skin cream, a cleanser, an exfoliator, a moisturizer, a sunscreen, a toner, a gel, a patch, and a healing cream or ointment.

20. The composition of claim 18, wherein multiple deficiency-based cosmeceutical formulations tailored to address various nutrient deficiencies are included together with the base cosmeceutical formulation in the cosmeceutical formulation.

21. The composition of claim 13, wherein the cosmeceutical formulation is one of a plurality of pre-formulated cosmeceuticals.

22. The composition of claim 21, wherein the pre-formulated cosmeceuticals comprise a pharmaceutical selected from the group consisting of:
High dose vitamins+High dose anti-oxidants+RDA dose minerals;
High dose vitamins+RDA dose anti-oxidants+RDA dose minerals;
RDA dose vitamins+High dose anti-oxidants+RDA dose minerals;
RDA dose vitamins+High dose anti-oxidants+High dose minerals;
High dose vitamins+High dose anti-oxidants+Amino Acids;
High dose vitamins+RDA dose anti-oxidants+Amino Acids;
RDA dose vitamins+RDA dose anti-oxidants+High dose minerals+Amino Aids.

23. The composition of claim 13, wherein the cosmeceutical formulation is formulated on a per individual basis, whereby each individual's cosmeceutical formulation is determined based on the individual's nutritional deficiencies.

24. A method comprising:
providing a personalized nutrient screening to identify nutrient deficiencies;
correlating the observed deficiencies to a database of growth factors to determine a set of growth factors that may be administered to address the deficiencies; and
providing a cosmeceutical with at least one active ingredient to address the deficiencies;
wherein the active ingredient comprises at least one vitamin, mineral, growth factor, peptide fragment of growth factors, antioxidant, or amino acid;
wherein the cosmeceutical is prepared from a base cosmeceutical formulation and a plurality of deficiency-based cosmeceutical formulations tailored to address various nutrient deficiencies.
